(19) United States
(12) Patent Application Publication
(10) Pub. No.: US 2009/0318986 A1
(43) Pub. Date: Dec. 24, 2009

(54) Systems, Methods and Apparatus for Treating Cardiac Dysfunction with Neurostimulation

(76) Inventors: Kenneth M. Alo, Houston, TX (US); Guillermo Torre-Amione, Houston, TX (US)

Correspondence Address: LOCKE LORD BISSELL & LIDDELL LLP;ATTN: IP DOCKETING, 600 TRAVIS, SUITE 3400, HOUSTON, TX, 77002-3095, US

(21) Appl. No.: 12/489,379

(22) Filed: Jun. 22, 2009

Related U.S. Application Data
(60) Provisional application No. 61/074,292, filed on Jun. 20, 2008.

(51) Int. Cl. A61N 1/365; A61N 1/36; A61N 1/362; A61N 1/39

(52) U.S. Cl. 607/4; 607/44; 607/9; 607/18; 607/17

(57) ABSTRACT

Methods, systems, and apparatus for the treatment of heart failure (both systolic and diastolic), hypertension, and arrhythmia in patients by stimulating one or more nerves, particularly peripheral nerves, using neurostimulation are described. The therapeutic treatment is accomplished by applying electrical signals to at least one or more nerves using cutaneous, subcutaneous, implantable, or catheter-based neurostimulation assemblies, alone or in combination with one or more additional therapy or stimulation devices associated with the patient's heart, and/or with one or more therapeutic drug infusions or therapies, such as immune modulation therapy (IMT).

### CROSS REFERENCE TO RELATED APPLICATIONS

The present application claims priority to U.S. Provisional Patent Application Ser. No. 61/074,292, filed Jun. 20, 2008, the contents of all of which are incorporated herein by reference in their entirety.

### STATEMENT REGARDING FEDERALLY SPONSORED RESEARCH OR DEVELOPMENT

Not applicable.

### REFERENCE TO APPENDIX

Not applicable.

### BACKGROUND OF THE INVENTION

1. Field of the Invention

The inventions disclosed and taught herein relate generally to methods, devices, and systems for treating cardiac dysfunction. More specifically, the inventions disclosed herein are related to methods, devices, and systems for treating cardiac dysfunction using neurostimulation for the treatment of cardiac mechanical dysfunction such as diastolic heart failure, systolic heart failure, arrhythmias, and hypertension.

2. Description of the Related Art

Heart failure (HF), which is generally characterized by impaired cardiac function and exercise intolerance, is an extremely serious affliction that affects a very large number of people worldwide, particularly in the Western world. Heart failure and its complications are responsible for premature death in a proportion of sufferers and generally curtails the working life and range of activities which can be undertaken by the sufferer, as well significantly reducing overall quality of life. Heart failure is found in both sexes, young and old but is particularly prevalent in males and elderly or middle aged people.

Heart failure is not a specific disease, but rather a compilation of ailments and symptoms, all of which are may be caused by a number of different underlying heart diseases which result in the inability of the heart to appropriately increase cardiac output during exertion. Heart diseases and events which may be a factor in causing chronic heart failure include valvular heart disease, valvular stenosis, heart muscle disease, myocardial ischemia or myocardial infarction, dilated cardiomyopathy, hypertrophic cardiomyopathy, restrictive/constrictive cardiomyopathy, and infiltrative process or inflammatory process of either the muscle, endocardium or epicardium of the heart. HF may also be caused by chronic hypertension, tachyarrhythmias, infarction or idiopathic cardiomyopathy. Chronic heart failure has a great impact on the quality of life; the sympathetic nervous system is placed into a state of hyperexcitablity leading to a loss of heart rate variability and rate responsive mechanisms in the heart. The ability of the heart to relax is impaired resulting in elevated filling pressures, pulmonary congestion and low exercise tolerance. These are just a few of the side effects. The classical symptoms of the disease include shortness of breath, edema, and overwhelming fatigue. As the disease progresses, the lack of cardiac output may contribute to the failure of other body organs, leading to cardiogenic shock, arrhythmias, electromechanical dissociation, and ultimately death.

As heart failure is a common and serious condition, significant efforts have been made by the medical community towards developing treatments for heart failure. A successful treatment should improve quality of life, prevent or slow progression of cardiac dysfunction and prolong life. While most of the approaches to the treatment of heart failure been centered around drug therapy regimes, several non-pharmacological treatments are used as well, including modified diets to reduce sodium retention and cause weight loss and exercise programs. However, there is a conflict between the need to improve ventricular performance that is aided by bed rest and a desire to improve exercise intolerance and maintain conditioning which is favored by a moderate exercise regime. Other approaches to the treatment of heart failure include treatment by surgical means, including full heart transplantation.

As indicated above, the primary method of treatment of severe cardiac dysfunction and decompensated heart failure is the application of drug therapies. number of pharmaceuticals are available for the treatment of heart failure and for the most part these fall into three broad categories-diuretics, vasodilators and inotropic drugs. Diuretic therapy seeks to maintain intravascular volume at the lowest level compatible with optimal cardiac performance. A reduction in intravascular volume has the advantage of reducing interstitial fluid by allowing its re-absorption into the vascular space. Furosemide and/or metolazone have been used as diuretics in the treatment of heart failure, but the use of these and other diuretics may lead to an undesirable drop in intracellular potassium levels. Inotropic drugs, such as dopamine or epinephrine, while useful in some instances, must be used with caution, as such drugs can cause harm in DHF patients such as resulting in ischemia, elevated heart rate, or arrhythmias. Similarly, vasodilators, which lead to a decrease in cardiac blood pressure, directly affects the relationship between mean arterial pressure and cardiac output and the total peripheral resistance (TPR), and so must also be used with caution.

While a large number of pharmaceuticals are available to the physician for treating heart failure, different patients will have different needs and successful treatment will often require administration of a range of complementary drugs, or have a number of undesirable side effects. Adverse reactions by some patients to particular drugs and drug intolerance means there is a continuing demand for new drugs of use in the treatment of heart failure, as physicians strive to find the best drug or combination of drugs for each sufferer. For example, ACE inhibitors have side-effects such as for example hypotension, persistent dry cough, gastrointestinal disturbances, taste disturbances, hyperkalaemia, acute renal failure, skin rashes, angioedema, and blood disorders. Moreover, heart disease is so widespread that the public and doctors alike demand ever more effective methods of treatment which can provide a higher quality of life for longer periods. One of the approaches that have been advanced of late is the use of electromechanical shock or stimulation therapy, such as artificial pacemakers and other implantable devices, such as AICD devices. An implantable cardioverter defibrillator (AICD) is a device that is implanted in the chest to monitor for and, if necessary, correct episodes of rapid heartbeat. If the heartbeat gets too fast (ventricular tachycardia), the AICD will stimulate the heart to restore a normal rhythm (anti-tachycardia pacing). In cases where the heartbeat is so rapid that the person may die (ventricular fibrillation), the AICD will also give an electric shock (defibrillation) to “reset” the heartbeat.

While a number of approaches have been made to affect treatment of patients suffering from cardiac disorders, the prior art electro-stimulation systems have not achieved a comprehensive therapy regimen that coordinates these mechanisms in a manner that is most effective without some degree of initiating potential arrhythmia with delivering a stimulation therapy, the problems typically arising from timing control issues. Further, numerous of the current devices which can be implanted into patients are separate devices which are not able to interact with each other, and which carry the potential to interfere with the operation of one another. For example, the devices may not interact, and give false positive signals to another implanted device, which could in turn lead to one of the devices shutting off, putting the patients life in danger.

One such system is described in U.S. Pat. No. 5,213,098, which discloses the use of post extrasystolic potentiation (PESP) in a manner that utilizes one or more sensors and signal processing circuitry to control timing parameters. For example, sensed physiologic signals are used to control the frequency or number of heart cycles between the delivery of one or more additional non-refractory pacing pulses. More specifically, a first sensor such as a ventricular or arterial blood pressure or flow sensor is employed to monitor the performance of the heart and to develop a cardiac performance index (CPI). A second sensor such as an oxygen saturation sensor positioned in the coronary sinus is employed to monitor cardiac muscle stress and develop a cardiac stress index (CSI). CPI and CSI are used to govern PESP stimulation application and timing to balance performance and stress. The disclosed PESP stimulator reportedly may be incorporated into a dual chamber pacing system with or without physiologic rate control (e.g., DDD). However, a problem associated with such methods using PESP is that the added ventricular depolarization may cause the loss of AV conduction during the next cardiac cycle. This in turn may result in loss of the next intrinsic depolarization in the ventricle, a problem that is commonly referred to as 2:1 AV block. The resulting pattern may be unstable, characterized by intermittent shifts between 2:1 and 1:1 conduction which may offset the other benefits provided by the PESP since ventricular filling is compromised.

What is needed is a system and/or method that provides for treating cardiac dysfunctions including heart failure (HF), in a manner that optimizes mechanical function or cardiac output, while also minimizing any risks to the patient associated with possibly inducing an arrhythmia or the like.

The inventions disclosed and taught herein are directed to methods, systems and associated devices (particularly devices having electrical separateness) for using neurostimulation methods for treating cardiac dysfunctions, as well as subsequent issues associated with heart failure, including arrythmias, hypertension, systemic problems, ischemia, and chest pain.

### BRIEF SUMMARY OF THE INVENTION

In brief, this application describes therapeutic treatment methods and associated systems for cardiac dysfunctions, such as heart failure and related cardiac events, using focused neurostimulation techniques and associated devices, so as to lower cardiac blood pressure, lower pulmonary vascular resistance (PVR), increase C.O. (cardiac output), and/or increase metastolic blood flow (MBF).

In accordance with a first aspect of the present disclosure, a method of treating a cardiac dysfunction in a human patient is described, wherein the method comprises positioning at least one neurostimulation device adjacent a nerve of a patient, and delivering an electrical stimulation pulse to the nerve of the patient for a period of time sufficient to result in an alleviation of the cardiac dysfunction.

In accordance with a further aspect of the present disclosure, a method of treating systolic heart failure in a patient is described, wherein the method comprises positioning at least one neurostimulation device adjacent a peripheral nerve of a patient, and delivering an electrical stimulation pulse to the peripheral nerve of the patient for a period of time sufficient to result in an alleviation of the systolic heart failure as indicated by decreased pulmonary resistance in the heart.

In yet another aspect of the present disclosure, a method of treating diastolic heart failure in a patient is described, wherein the method comprises positioning at least one neurostimulation device adjacent a peripheral nerve of a patient, and delivering an electrical stimulation pulse to the peripheral nerve of the patient for a period of time sufficient to result in an alleviation of the diastolic heart failure as indicated by decreased blood pressure in the heart.

In a further aspect of the present disclosure, a method of treating arrhythmias in a patient is described, wherein the method comprises positioning at least one neurostimulation device adjacent a peripheral nerve of a patient; positioning at least one neurostimulation device adjacent a nerve in or near the brain of a patient; and delivering an electrical stimulation pulse to the peripheral nerve and the nerve near the brain of the patient for a period of time sufficient to result in an alleviation of the arrhythmia as indicated by decreased arterial fibrillation and/or ventricular fibrillation.

In yet another aspect of the present disclosure, a method of treating hypertension in a patient is described, wherein the method comprises positioning at least one neurostimulation device adjacent a peripheral nerve of a patient; positioning at least one neurostimulation device adjacent a nerve in or near the C2-C4 spinal region of a patient; and delivering an electrical stimulation pulse to the peripheral nerve and the nerve near the C2-C4 spinal region of the patient for a period of time sufficient to result in an alleviation of the hypertension as indicated by improved blood pressure control in the patient.

In a further aspect of the present disclosure, a system for treating cardiac dysfunction in a patient is described, wherein the system comprises a stimulator adapted to deliver a stimulation signal for a heart failure event; a plurality of sensors adapted to measure and provide information of a heart failure condition status at two separate times; and a controller in communication with the stimulator and the plurality of sensors and adapted to use the measurements from the sensors to control the stimulator to modulate the electrical signal. In further accordance with this aspect, the stimulator may comprise at least one neural stimulator adapted to deliver a neural stimulation signal to a neural target for heart failure therapy. In further accordance with this aspect of the disclosure, the information measured by the sensors includes an activity measurement, a blood pressure measurement, a pulmonary vascular resistance measurement, a heart rate variability (HRV) measurement, a heart rate turbulence (HRT) measurement, a respiration measurement, a heart sound measurement, a thoracic impedance measurement, and/or a metastolic blood flow measurement. In further accordance with this aspect of the disclosure, the system may be provided such that the stimulator is a neurostimulator, and the device further comprises one or more devices consisting of an implantable cardioverter defibrillator (AICD), a bi-ventricular (BiV) device, and at least one feed-back loop, wherein the devices have electrical separateness (meaning that there isn't interference between devices, and the devices can interact simultaneously).

In yet another aspect of the present disclosure, a system for treating cardiac dysfunction in a patient is described, wherein the system comprises means for applying an electrical stimulation to one or more nerves; means for measuring one or more indicators of heart failure status in a patient; means for monitoring the information obtained by the measurement means; and means for adjusting the intensity and rate of application of electrical impulses using the information obtained by the measurement means.

### BRIEF DESCRIPTION OF THE SEVERAL VIEWS OF THE DRAWINGS

The following figures form part of the present specification and are included to further demonstrate certain aspects of the present invention. The invention may be better understood by reference to one or more of these figures in combination with the detailed description of specific embodiments presented herein.

FIG. 1 illustrates a dermatome map of the human body, with each dermatome region corresponding to a longitudinal spinal position.

FIG. 2 illustrates an isometric view of a conventional spinal cord stimulation lead, in accordance with aspects of the present disclosure.

FIG. 3 illustrates a superior view of the spinal cord in the vertebral canal, illustrating points 200 where neurostimulation leads can be placed in accordance with the present disclosure.

FIG. 4 illustrates a number of exemplary neurostimulation leads which may be used in the methods and systems of the present disclosure.

While the inventions disclosed herein are susceptible to various modifications and alternative forms, it is to be realized that only a few specific embodiments have been shown by way of example in the drawings and are described in detail below. The figures and detailed descriptions of these specific embodiments are not intended to limit the breadth or scope of the inventive concepts or the appended claims in any manner. Rather, the figures and detailed written descriptions are provided to illustrate the inventive concepts to a person of ordinary skill in the art and to enable such person to make and use the inventive concepts.

### DEFINITIONS

The following definitions are provided in order to aid those skilled in the art in understanding the detailed description of the present invention.

The term “arrhythmia” as used herein means an abnormal heart rhythm in a patient.

The term “cardiac ischemia” refers to heart conditions wherein the myocardium is deprived of adequate oxygen and metabolite due to an interruption in blood supply caused by an occlusion of a blood vessel, such as a coronary artery.

The term “congestive heart failure” (CHF), as used herein, refers to a clinical syndrome due to heart disease, characterized by breathlessness and abnormal sodium and water retention, often resulting in edema. The congestion may occur in the lungs or peripheral circulation or both, depending on whether the heart failure is right-sided or general.

The term “diastolic heart failure”, or “DHF”, as used herein, refers to heart failure conditions that are due to a defect in ventricular filling caused by an abnormality in diastolic function, or fluid pumping pressure, of the heart, such that there is an abnormality in the heart's ability to handle the elevation of pressure within the heart, while the pumping pressure is preserved.

The term “systolic heart failure”, or “SHF”, means heart failure due to a defect in expulsion of blood caused by an abnormality in systolic function, or pumping pressure resulting from weak heart muscles, such that the decrease in pumping function associated with the weak pumping pressure, prevents the pumping of blood from the heart. This in turn leads to elevated pressure due to the weakened heart muscle.

The term “hypertension” as used herein refers to a medical condition in which the blood pressure is chronically elevated, and which may be classified as either essential (primary) hypertension or secondary hypertension.

The term “myocardial infarction”, or “MI”, refers to the necrosis of portions of the myocardial tissue of the heart resulting from cardiac ischemia.

As used herein, the term “RAS” refers to Renin Angiotensin System, a chemical system within the body made up of renin (an enzyme produced in the kidneys that acts on angiotensinogen, produced in the liver, and forms angiotensin I), ACE, and angiotensin II.

As used herein, the terms “subject” and “patient” are used interchangeably. The terms “subject” and “subjects” (or, “patient” and “patients”) refer to an animal, preferably a mammal including but not limited to a non-primate (e.g., a cow, pig, horse, cat, dog, rat, and mouse) and a primate (e.g., a monkey such as a cynomolgous monkey, a chimpanzee and a human), and more preferably a human. In one embodiment, the subject is refractory or non-responsive or minimally-responsive to current treatments for a disorder (e.g., a disorder characterized by aberrant heart rates and/or blood pressures). In a preferred embodiment, the subject is a human.

The term “neurostimulation”, as used herein, refers to electrical intervention (transcutaneously or by way of an inserted stimulation apparatus) into a mammalian body for a therapeutic or diagnostic purpose. Neurostimulation includes, but is not limited to, electrical (AC, DC, AC/DC, and the like) therapy; radiofrequency (including both pulsed and non-pulsed) therapy; spinal cord stimulation (SCS); distal spinal column stimulation (DCS); spinal nerve root stimulation (SNRS); sacral nerve stimulation; intradiscal electrothermal therapy; radiofrequency thermocoagulation; chemical intervention; mechanical intervention; or a combination thereof to modulate or change central and peripheral nervous system function for the treatment of cardiac disorders and dysfunction, in adult patients. Neuromodulation, as used herein, can also broadly encompass the modulation of the effects of a specific receptor or ion channel on neuronal excitability by the activation of a different receptor or channel, in an acute or chronic nature.

As used herein, the terms “treat”, “treatment” and “treating” refer to the reduction or amelioration of the progression, severity and/or duration of a disorder (e.g., a disorder characterized by a heart-related effect, such as an increase or decrease in blood pressure, or a CHF-related effect or disorder), or the amelioration of one or more symptoms thereof resulting from the administration of one or more therapies or therapeutic methods (e.g., one or more therapeutic methods such as one or more of the neurostimulation procedures of the instant disclosure). In specific embodiments, such terms refer to the inhibition or reduction in the symptoms associated with a congestive heart failure (CHF) event, the inhibition or reduction in the onset, development or progression of one or more symptoms associated with CHF, the reduction in the size of the heart, or the improvement in a patient's acute physiology score. In yet other embodiments, such terms refer to a reduction a human's daily living score or an improvement in a human's Minnesota Living with Heart Failure Questionnaire (MLHFQ) score.

### DETAILED DESCRIPTION

The Figures described above and the written description of specific structures and functions below are not presented to limit the scope of what Applicants have invented or the scope of the appended claims. Rather, the Figures and written description are provided to teach any person skilled in the art to make and use the inventions for which patent protection is sought. Those skilled in the art will appreciate that not all features of a commercial embodiment of the inventions are described or shown for the sake of clarity and understanding. Persons of skill in this art will also appreciate that the development of an actual commercial embodiment incorporating aspects of the present inventions will require numerous implementation-specific decisions to achieve the developer's ultimate goal for the commercial embodiment. Such implementation-specific decisions may include, and likely are not limited to, compliance with system-related, business-related, government-related and other constraints, which may vary by specific implementation, location and from time to time. While a developer's efforts might be complex and time-consuming in an absolute sense, such efforts would be, nevertheless, a routine undertaking for those of skill this art having benefit of this disclosure. It must be understood that the inventions disclosed and taught herein are susceptible to numerous and various modifications and alternative forms. Lastly, the use of a singular term, such as, but not limited to, “a,” is not intended as limiting of the number of items. Also, the use of relational terms, such as, but not limited to, “top,” “bottom,” “left,” “right,” “upper,” “lower,” “down,” “up,” “side,” and the like are used in the written description for clarity in specific reference to the Figures and are not intended to limit the scope of the invention or the appended claims.

Particular embodiments of the invention may be described below with reference to block diagrams and/or operational illustrations of methods. It will be understood that each block of the block diagrams and/or operational illustrations, and combinations of blocks in the block diagrams and/or operational illustrations, can be implemented by analog and/or digital hardware, and/or computer program instructions. Such computer program instructions may be provided to a processor of a general-purpose computer, special purpose computer, ASIC, and/or other programmable data processing system. The executed instructions may create structures and functions for implementing the actions specified in the block diagrams and/or operational illustrations. In some alternate implementations, the functions/actions/structures noted in the figures may occur out of the order noted in the block diagrams and/or operational illustrations. For example, two operations shown as occurring in succession, in fact, may be executed substantially concurrently or the operations may be executed in the reverse order, depending upon the functionality/acts/structure involved.

Applicants have created systems, devices and methods for providing neural stimulation to a patient in order to treat one or more cardiac dysfunctions, such as diastolic heart failure, systolic hear failure, arrhythmias, and similar ischemic events.

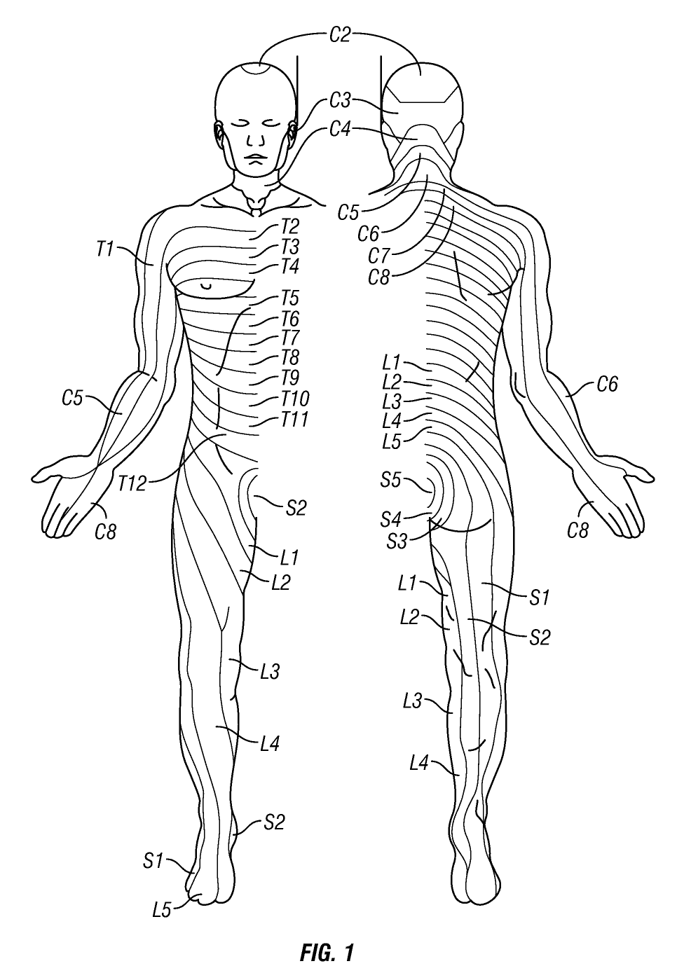

Turning now to the figures, FIG. 1 is an illustration of a dermatome map of the human body, with each dermatome region corresponding to a longitudinal spinal position. It is known that each exterior region, or each dermatome, of the human body is associated with a particular spinal nerve root at a particular longitudinal spinal position. As shown schematically in FIG. 1, the head and neck regions (including the brain) are associated with C2-C8, the back regions extend from C2-S3, the central diaphragm is associated with spinal nerve roots between C3 and C5, the upper extremities correspond to C5 and T1, the thoracic wall extends from T1 to T1, the peripheral diaphragm is between T6 and T1, the abdominal wall is associated with T6-L1, the lower extremities are located from L2 to S2, and the perineum from L4 to S4 (with S1-S5 being the sacral region). By example, to address chronic pain sensations that commonly focus on the lower back and lower extremities, a specific energy field can usually be applied to a region between bony level T8 and T10. As should be understood, successful pain management and the avoidance of stimulation in un-afflicted regions necessarily requires the applied electric field to be properly positioned longitudinally along the dorsal column.

### Therapeutic Targets and Methods.

The therapeutic targets associated with cardiac dysfunction which may be treated with neurostimulation therapy in accordance with the present disclosure include SHF, DHF, hypertension, and arrhythmias. The methods generally comprise selective placement of one or more neurostimulation leads in an appropriate location on the patient's body, such as epidurally, laterally, or upside-down in the pelvis area, so as to send stimulation to the appropriate regions of the body and treat a cardiac disorder or dysfunction associated with CHF.

In a first embodiment of the present disclosure, methods are described for the treatment of systolic heart failure (SHF) using neurostimulation techniques. In accordance with this method, an electrical impulse signal is sent to the heart, while simultaneously an electrical stimulus is begun in a peripheral region of the patient, such as at T4, so as to cause vasodilation of the heart of the patient to increase the C.O., and decrease the PVR (pulmonary vascular resistance), while simultaneously allowing the metastatic blood flow rate of the patient's heart to increase. In accordance with this method, it is envisioned that the neurostimulation signals may be applied in near 100% capacity during the day, and then reduced to about 50% capacity during the night.

In a further embodiment of the present disclosure, methods are described for the treatment of diastolic heart failure (DHF) using neurostimulation. In accordance with this method of the disclosure, a neurostimulation signal is applied near the brain region a patient, such as near the carotid sinus, and simultaneously to the heart, so as to vasodilate the heart, and result in a decrease of blood pressure in and around the heart, which in turn counter-acts diastolic heart failure.

In another embodiment of the present disclosure, methods are described for the treatment of hypertension in a human patient using neurostimulation. As described herein, hypertension refers to pulmonary hypertension (e.g., hypertension associated with heart failure); as well as primary hypertension (also known as essential, or idiopathic hypertension, and referring to a type of hypertension having no identifiable cause), and secondary hypertension (less commonly, inessential hypertension, a type of hypertension caused by an identifiable, underlying secondary cause, including renal diseases and disorders, endocrine diseases and disorders, kidney diseases and disorders, tumors, and as a side effect to certain medications.). In accordance with this method of the disclosure, a neurostimulation signal is applied to both the cranial (brain) region of the patient as well as to a peripheral region of the patient, so as to reduce or advantageously affect the heart and reduce the symptoms of hypertension. For example, it is envisioned that, using subcutaneous and/or epidural electrodes placed at or adjacent the C2 region of the patient's spine and at or adjacent one or more of the T1-T4 nerve regions (corresponding to the heart) of the patient, a decrease in hypertension and a gradual return of the heart to normal may be achieved.

In yet another embodiment of the present disclosure, methods are described for the treatment of arrhythmias in a human patient using neurostimulation. In accordance with this method of the present disclosure, it is envisioned that one or more neurostimulation signals are applied to the central nerve region of the patient, to the cranial region of the patient, and at one or more peripheral locations (e.g., T9) within the patient, so as to decrease the occurrence of arrhythmias in the patient. In further accordance with this aspect, the neurostimulation electrodes being placed peripheral to, cutaneously, and/or subcutaneously on, in, or adjacent these regions of the patient, can result in decreased arterial and ventricular fibrillation within the patient.

The methods for neurostimulation/neuromodulation disclosed above, and in accordance with the present disclosure, comprise implanting (subcutaneously, peripherally, spinally, centrally, cutaneously, percutaneously, or a combination thereof) one or more electrodes in a target area of the patient to be stimulated. Typically, the electrodes can be implanted in any appropriate space, such as the epidural space between the vertebrae and the dura, as shown in FIG. 3. Such implants can be located centrally (200a, 200d), or can be located in the lateral gutter (200b, 200c) of the epidural space. These placements can be done for a number of reasons, including reduced complexity of the procedure, reduced potential complications, increased stability of the implant, and improved clinical efficacy, to name a few. Neurostimulation leads can be implanted in the spinal region (at any spinal level or position within the canal relative to the nervous or musculoskeletal tissue) of the body, or in peripheral areas of the nervous system, using techniques known to those of skill in the art, including but not limited to anterograde implantation techniques, retrograde implantation techniques, subdural techniques, combinations of such techniques, and the like. Such methods and techniques include, but are not limited to, those methods and techniques described and referenced in U.S. Pat. Nos. 6,553,264; 6,309,401; 6,587,733; 6,104,957; and 6,002,964. Because the inventions of the present disclosure are useful in the treatment of cardiac disorders, the stimulation leads (and necessarily, the associated feedback and sensory leads), can also, or alternatively, be placed in one or more regions of the heart itself, as atrial or ventrical leads (e.g., in the right ventrical, left ventrical, or both the right and left ventrical, and/or as or with a circular lead around the heart itself.

### Leads for Electrical Stimulation.

The stimulation leads/catheters suitable for use in the neurostimulation methods and processes disclosed herein include a variety of known leads and electrodes, optionally modified as necessary to be useful in the applications described herein to treat cardiac dysfunctions such as SHF, DHF, hypertension, and arrhythmias. Such leads include, but are not limited to, implantable leads; transdermal leads; ventrical or atrial leads; circular leads (such as leads that substantially circumscribe the heart); percutaneous leads; single, tubular axial leads; lead assemblies comprising two or more lead bodies bonded together; paddle-style leads; multi-electrode leads, having from 2 to any suitable multiple of 2 (e.g., 4, 8, 16, 24, 32, 64, 128, and the like) electrodes; multi-probe, multi-functional catheter systems comprising a plurality of axial lumens; winged leads; ceramic-based, multi-site leads; low impedance leads; coated leads; leads comprising multiple channels or passages for the introduction of drugs, scopes (working or video or other), thermal, or photonic energy as well as electrical energy to the anatomical region to be stimulated; implantable stimulators; pulsed radiofrequency leads; external electronic stimulation devices, such as those utilized in transcutaneous electric neurostimulation (TENS); leads capable of using DC current, leads adapted for pulsed electromagnetic stimulation (PEMS); and combinations of such leads. Examples of suitable leads for use in accordance with the present disclosure include, but are not limited to, percutaneous leads such as the OCTRODE® lead manufactured by Advanced Neuromodulation Systems, Inc.; low impedance leads; paddle leads such as the LAMITRODE™ series of paddle-type leads; trial leads such as the QUATRRODE® lead; cochlear implant leads, such as those available from Bion microstimulators (Advanced Bionics); extended life neurostimulation leads (Medtronic Neurological); implantable generators and leads having current steering means; implantable stimulators, small enough to be located near or within an area of the spine responsible for sensations in a region experiencing chronic pain uses a power source/storage device, such as a rechargeable battery; stimulation prostheses that include (1) an implantable hermetically sealed case wherein electronic circuitry, including a battery and an implantable microphone, are housed, (2) an active electrode array that provides a programmable number of electrode contacts through which stimulation current can be selectively delivered to surrounding tissue, preferably through the use of appropriate stimulation groups, and (3) a connector that allows the active electrode array to be detachably connected with the electronic circuitry within the sealed case; and all other leads, electrodes, and the like in current use in the field of neuromodulation and neurostimulation therapy.

The size, shape, length, configuration, orientation, electrical field density, number of contact electrodes, and material that the leads are made of can vary, depending upon the specific therapeutic utility and optimal placement desired. For example, the leads can be circumferential or non-circumferential, have any length, as determined by the specific use of the lead and the disorder to be treated, as well as the method of treatment. For example, in one aspect, the length of the lead can range from less than 1 mm to a length of 100 cm or more. In other aspects of the present invention, the lead length can range from about 1 mm to about 20 mm, although lengths less than 1 mm, such as nanotube-type leads, can also be used. Similarly, the length between electrodes can vary as appropriate, ranging, for example, from about ¼ mm to about 10 mm or greater (e.g., ranging from about 0.001 to 50 cm, and all lengths in between), depending upon the use and application of the lead. Additionally, the specific orientation radius of the electrode about the lead can range from about 30° to about 360°, including 60°, 90°, 180°, 270° and radii in between these values.

The leads suitable for use with the present disclosure can be made from a variety of suitable materials, including but not limited to metals such as surgical steel, metal alloys, transition metals of the Periodic Table, such as titanium, transition-metal alloys, plastic and other suitable polymers such as polyvinyl chloride, and combinations thereof. Additionally, the lead or leads can further comprise a sheath. Such sheath can include, for example, fixing elements configured to fix the electrode(s) of the lead along the tissue in the appropriate spot. The fixing element can include, but is not limited to, inflatable balloons, nitinol, tines, and sheath shape. Further, one or more optical channels can be provided and extend from a port at the proximal end of the lead or sheath to a port at the distal end of the lead or sheath, inside the body. The port for the optical channel at the distal end can be located at one or more spaces at the distal end of the lead, or in an area or areas between electrodes. The presence of such an optional optical channel can provide photonic energy to the tissue while simultaneously functioning as a lens for a remote camera useful in assisting in the placement of the lead(s). Examples of leads suitable for use in the present invention, while in no means meaning to be limiting, are shown in FIG. 6, and include both rechargeable and non-rechargeable variants thereof. Leads suitable for use with the present invention can also include, for example, those leads having electrodes that fuse the electrode(s) and the power (battery) source into one or more micro-type designs, such as the Bion® microstimulator by Advanced Bionics, forming a bi-polar device that is rechargeable and only a few inches in length. Also suitable for use herein are those leads having multiple-contact electrodes.

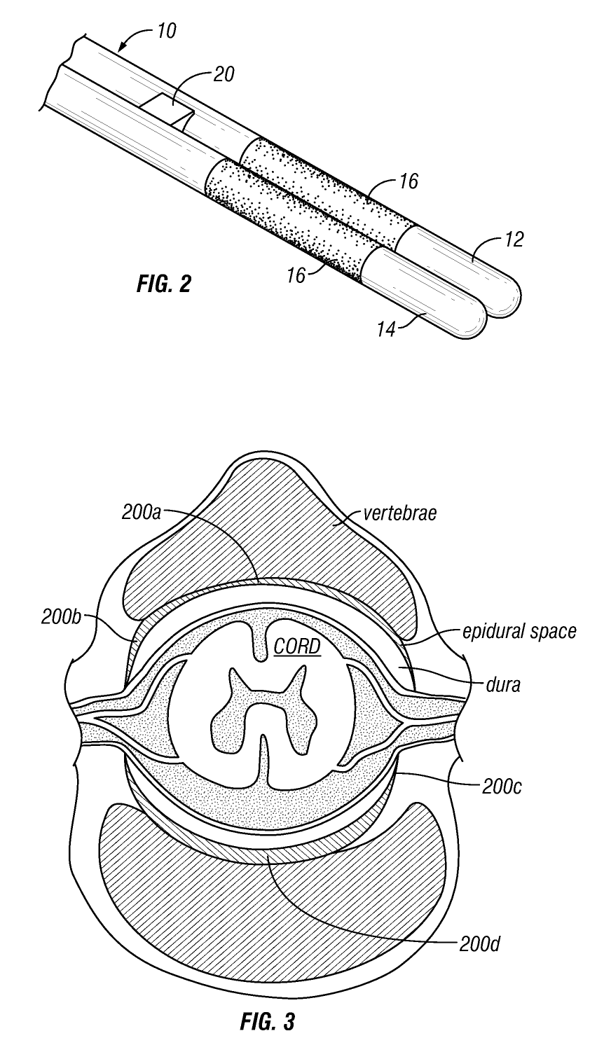

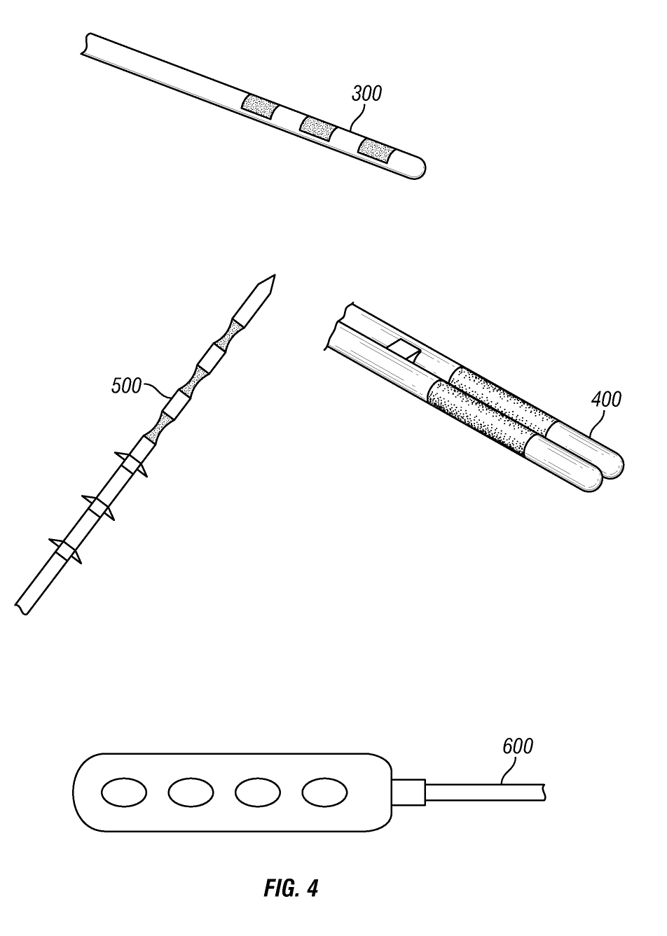

Examples of suitable leads which can be used or modified as necessary according to aspects of the present invention include but are not limited to those leads described in, for example, U.S. Pat. Nos. 6,999,819; 6,895,280; 6,587,733; 6,309,401; 6,553,264; 5,423,877; 5,374,285; 5,081,990; 4,919,140; 4,379,462; and WO 92/07605, as well as references cited and referenced therein, all of which are incorporated herein by reference. Examples of leads suitable for use in the present invention, while in no means meaning to be limiting, are illustrated in FIG. 2, which illustrates an enlarged, isometric view of a conventional, percutaneous spinal cord stimulation lead 10 comprising a lead bodies 12 and 14 in a parallel arrangement with electrodes 16 have a controlled orientation on the lead bodies (similar to those described and claimed in U.S. Pat. No. 6,587,733 and U.S. Patent Publication No. 2003/0229387, both of which are incorporated herein by reference); and, in FIG. 4 as leads 300, 400, 500 and 600.

Further, all of the apparatus and assemblies suitable for use herein, including the delivery devices for the delivery of electrical or other suitable stimulation means, can include both those known in the art, and improvements made thereto for specific use in the population encompassed by this disclosure (e.g., pregnant mothers, fetuses’, neonates, small children, adolescents, and pre-adults). Such stimulus delivery devices can comprise an energy storage device, one or more visual training systems (e.g., to monitor the implantation or insertion of the apparatii), combinations of stimulus delivery devices that are the same or different (e.g., an electrical delivery device and an radio-frequency delivery device), and the like. The devices can include pulse generating (e.g., electrical pulse) means, such as battery operated and programmable devices. Also incorporated herein are those apparatus useful in neuromodulation that comprise a drug pump, which can be battery operated, programmable, comprising a membrane or no-membrane, or a combination of both, useful for administering one or more drugs to a space within the body. The device can be programmed in accordance with a variety of methods known in the art, and can be current controlled, voltage controlled, impedance controlled, etc., and can also include internal pulse generators, external pulse generators, and combinations thereof. Similarly, the devices can be rechargeable or non-rechargeable, have a battery or other suitable energy source, and can be meant for permanent or “trial”/temporary implantation. The devices may also be capable of remote operation, or control, using such technology as Bluetooth technology and other wireless communication devices. Further, as the devices, systems, and methods of the present disclosure are designed to address a plurality of disease scenarios, e.g., two or more disease states, such as a cardiac dysfunction and a neurological-related disorder, at different stages, the device may also include one or microprocessors capable of learning and self-evolving, including but not limited to neural networks, learning processors, look-up tables, and other self-evolving, trainable devices. In further accordance with the disclosure, the energy used for stimulation in accordance with the present disclosure includes electrical energy/current (such as AC, DC, or a combination of AC and DC energy), radiofrequency energy/current, pulsed radiofrequency energy/current, acoustic energy/current, light energy, and combinations thereof.

### Monitoring.

Measurement of the therapeutic treatments for HF using the neurostimulation techniques, methods, and systems described herein include, but are not limited to, objective measuring tools and methodologies, such as EKGs and similar devices; heart rate feedback sensors; end-point markers, such as bmp serological markers; heart and blood flow pressure sensors; thorasic fluid content monitors; cardiodynamic monitors; blood pressure sensors; blood flow rate sensors; and blood hormone concentration sensors, as well as combinations of such monitoring systems, techniques, and methods.

Control and measurement of the treatments using the devices and systems described herein may also be through the use of one or more feedback loops for functionalstimulation. Exemplary, non-limiting feedback loops include bladder stimulators, which give feedback as to bladder tone, illustrating that the feedback loops suitable for use herein can be designed to be specific to the organ or organs that they are associated with, while simultaneously not interfering with other devices implanted in the patient, such as pacemakers and the like. Additional, exemplary feedback loops suitable for use herein include loops with sensors located in any number of cardiac spaces, such as in the atrium. The devices and feedback loops could be intermittent, or could be constantly on, as necessary. In example, in accordance with the present disclosure, a method of treatment would be to use neurostimulation to prevent atriofibrulation in a patient, or interrupt atriofibrulation using electrical neurostimulation, with a coronary sinus sensor and another elsewhere in the patient's body, which then turns on a device located higher along the patient's spine, and then turns on the stimulator on or in the coronary sinus (between the inferior vena cava and the auriculo-ventricular opening). The spinal neurostimulator could also be always on, if appropriate, wherein the intra-atrial sensor could be activated in by stimuli in or near the spinal area, or subcutaneosly. Such an exemplary treatment combination in association with the present disclosure may be classified as both prevention and treatment methodologies.

### Drug Therapy.

In accordance with certain aspects of the present disclosure, the methods described herein for use in the treatment of cardiac dysfunction may optionally include, or be performed in combination with, the administration of one or more therapeutic agents. That is, as devices and systems of the present disclosure may have electrical separateness so that there isn't interference between implanted devices, there may be a device in the field that runs non-interfering applications, e.g., a drug-delivery device in accordance with neurostimulation.

Suitable therapeutic agents for use in association with the methods of the present disclosure include, but are not limited to, those therapeutic agents which treat, or contribute to the alleviation of the symptom of, the cardiac dysfunction, such as by lowering pulmonary pressure, relaxing the heart muscles, lowering pulmonary (lung) pressures, and relaxing the arteries (vasodilation). In particular, the therapeutic agents which may be used in combination or association with the methods and systems described herein include inotropic therapeutic agents, nitrates, digoxins, loop diruretics, thiazide diuretics, beta blockers (β-blockers), calcium channel blockers, and RAS inhibitors, alone or in combination. Also suitable for use with the systems and methods described are anti-inflammatory agents, genetically modified modalities, such as IMT and the like, and gamma-aminobutyric acid-B (GABAB) receptor agonists such as Baclofen, all of which may be used alone or in combination with the heart-related therapeutic agents recited and discussed herein.

Inotropic therapeutic agents are those therapeutic agents which have the characteristic of causing a patient's heart to beat more strongly. Inotropic therapeutic agents suitable for use in association with the systems and methods of the present disclosure include, but are not limited to, both positive inotropes (agents that increase myocardial contractility) and negative inotropes (agents decrease myocardial contractility), the former group including groups of compounds such as β-adrenergic agonists, eicosanoids, and phosphodiesterase-III inhibitors, and negative inotropes including diltiazem and verapamil. Beta-adrenergic agonists suitable for use as inotropic therapeutic agents within the present disclosure include dobutamine, dopamine, and derivatives thereof. Phosphodiesterate-III inhibitors suitable for use as inotropic therapeutic agents include milrinone (Primacor) and aminone (Inocor).

Nitrates, in particular organic nitrates (esters of nitric acid) that are suitable for use as therapeutic agents in accordance with the present disclosure are those compounds that display their effect both by relieving the heart via a reduction in the preload and afterload and by improving the oxygen supply to the heart via coronary dilatation. Nitrates suitable for use herein include but are not limited to glycerol trinitrate, isosorbide dinitrate, isosorbide 5-mononitrate, and nitro compounds containing a disulphide group.

Loop diuretics suitable for use with the methods and assemblies of the present disclosure are diuretic agents that act on the ascending loop of Henle in the kidney, by acing on the Na⁺—K⁺-2Cl⁻ symporter (cotransporter) in the thick ascending limb of the loop of Henle to inhibit sodium and chloride reabsortpion. Exemplary loop diuretics include, but are not limited to, those agents typically used to treat hypertension and edema resulting from CHF. Exemplary loop diuretics suitable for use with the present disclosure include but are not limited to bumetanide (BUMEX®), furosemide (LASIX®), ethacrynic acid, and torsemide (DEMADEX®), alone or in combination.

Thiazide diuretics include the class of diuretics that are generally derived from benzothiadiazine. They inhibit Na⁺/Cl⁻ reabsorption from the distal convoluted tubules in the kidneys by blocking the thiazide-sensitive Na⁺—Cl⁻ symporter. Thiazides also cause loss of potassium and an increase in serum uric acid, and are often used to treat hypertension, congestive heart failure and symptomatic edema. Exemplary thiazide diuretics suitable for use in conjunction with the therapies of the present disclosure include but are not limited to Hygroton® (chlorothalidone), chlorothiazide, Hydrodiuril®, Esidrix (hydrochlorothiazide), Amiloride®, bendroflumethiazide, and Aldactone® (spironolactone).

Beta blockers, also referred to as beta-adrenergic blocking agents, beta-adrenergic antagonists, or beta-antagonists, are those agents which act to block the action of endogenous catecholamines (such as adrenaline and noradrenaline) on β-adrenergic receptors. Suitable β-blockers for use with the methods and systems of the present disclosure include but are not limited to non-selective β-blocking agents such as Alprenolol, Carteolol, Levobunolol, Mepindolol, Metipranolol, Nadolol, Oxprenolol, Penbutolol, Pindolol, Propranolol, Sotalol, and Timolol; β-selective agents such as Acebutolol, Atenolol, Betaxolol, Bisoprolol, Esmolol, Metoprolol, and Nebivolol; mixed α₁/β-adrenergic antagonists such as Carvedilol, Celiprolol, and Labetalol; and β₂-Selective agents, such as butaxamine (which exhibits weak α-adrenergic agonist activity).

Calcium channel blockers suitable for use in association with the methods and systems of the present disclosure include the class of drugs and natural substances which have an effect on excitable cells of the body, such as the muscle of the heart. In particular, it is preferred that the calcium channel blockers used herein include those compounds of this class that are capable of decreasing the force of contraction of the myocardium. Exemplary calcium channel blockers suitable for use with the present disclosure include, but are not limited to, dihydropyridines such as amlodipine (Norvasc®), nicardipine (Cardene®), nifedipine (Procardia®), nitrendipine, and lercanidipine; phenylalkylamines such as verapamil and gallopamil; and benzothiazepines such as diltiazem (Cardizem®).

RAS inhibitors suitable for use as therapeutic agents in association with the methods and systems of the present disclosure include both ACE inhibitors and ARBs, as well as aldosterone antagonists. ACE inhibitors are antihypertensive drugs that act as vasodilators and reduce peripheral resistance. They inhibit angiotensin converting enzyme (ACE), which is involved in the conversion of angiotensin I to angiotensin II. Angiotensin II stimulates the synthesis and secretion of aldosterone and raises blood pressure via a potent direct vasoconstrictor effect. ACE is identical to kininase II, an enzyme that inactivates bradykinin and other potent vasodilator peptides. ACE inhibitors may reduce the degradation and increase levels of bradykinin, a potent vasodilator. Exemplary ACE inhibitors suitable for use herein include but are not limited to Alacepril, Benazepril, Captopril, Ceronapril, Cilazapril, Delapril, Enalapril, Enalaprilat, Fosinopril, Imidapril, Lisinopril, Moexipril, Moveltipril, Perindopril, Quinapril, Ramipril, Spirapril, Temocapril and Trandolapril.

Angiotensin II Receptor Blockers (ARBs) are medications that block the action of angiotensin II. As a result, the blood vessels dilate and the blood pressure is reduced. The lower blood pressure makes it easier for the heart to pump blood and can improve heart failure. Exemplary ARBs include but are not limited to candesartan (ATACAND®), eprosartan (TEVETAN®), irbesartan (AVAPRO®), telmisartan (MYCARDIS®), valsartan (DIOVAN®), and losartan (COZAAR®). Aldosterone antagonists are those drugs which antagonize the action of aldosterone (a steroid) at mineralocorticoid receptors. Suitable members of this class which may be used in association with the present disclosure includes but is not limited to spirolactones, such as spirolactone, eplerenone, and canrenone, as well as derivatives, analogs, and metabolites thereof.

In yet another aspect of the present disclosure, one or more anti-inflammatory agents may be used in association with the neurostimulation methods and systems of the present disclosure in order to reduce inflammatory occurrences in the patients, and increase anti-inflammatory cytokines and aid in the therapeutic treatment of patients with heart failure (HF) by reducing the inflammation cascade. Generally speaking, any anti-inflammatory therapy (e.g., an anti-inflammatory therapeutic agent) well-known to one of skill in the art can be used in the compositions, methods and systems of the present disclosure. Non-limiting examples of anti-inflammatory agents sutable for use herein include but are not limited to non-steroidal anti-inflammatory drugs (NSAIDs), steroidal anti-inflammatory drugs, beta-agonists, anticholingeric agents, antihistamines (e.g., ethanolamines, ethylenediamines, piperazines, and phenothiazine), and methyl xanthines. Non-limiting examples of NSAIDs include, but are not limited to, aspirin, ibuprofen, salicylates, acetominophen, cuelecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxygenase enzyme (e.g., COX-1 and/or COX-2). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

In further accordance with the present disclosure, genetically modified modalities, such as IMT (immune modulation therapy, a therapy which triggers an immune response, which in turn improves cardiac function by reducing harmful inflammation) may be used in association with the neurostimulation methods and systems of the present disclosure in order to reduce inflammatory occurrences in the patients, and increase anti-inflammatory cytokines and aid in the therapeutic treatment of patients with heart failure (HF) by reducing the inflammation cascade. This resultant decrease in inflammation in the patient can help in stabilizing the medications that the patient is or may be taking, resulting in improved physical advancements post HF event. Exemplary IMT approaches suitable for use herein include the CELACADE™ technology being developed by Vasogen, Inc. (Mississauga, Ontario, Canada).

### Other Applications.

The methods, techniques, systems, and assemblies described herein may also be useful in a variety of other applications, outside of the treatment of CHF, arrhymias, and hypertension in patients. For example, and without limitation, it is envisioned that the neurostimulation methods and systems described herein may be useful in the treatment of post-operative atrofibrillation, a common cardiac arrhythmia that affects about 5% of the elderly patient population, is a very potent risk factor for stroke, and in some instances is a cardiac inflammation event associated with the use of warfarin (COUMADIN®, an anti-coagulant) in treatment regimens. The neurostimulation methods and systems described herein may also be useful in combination with the use of pain control and local anesthesia, such as through the use of a combined infusion/neurostimulation catheter, wherein the catheter includes a neurostimulation devices such as described above, as well as a local anesthetic, such as about 0.125% mepivacaine or an equivalent.

A further application of the methods and systems described herein includes the stimulation of the heart or epicardial vessels by placing a neurostimulation device directly on or near the hear or vessels in order to provide a therapeutic effect. Similarly, it is envisioned that neurostimulation electrodes may be placed in the myocardium, or in another appropriate location via endovascular placement, in order to enhance the efficiency of the heart. Such endovascular placements of neurostimulation electrodes may also enhance the efficiency and operation of pace-makers.

Another application of the methods and systems described herein includes use of these neurostimulation techniques to augment cardiac resynchronization therapy (CRT), acting to stimulate inside the heart so as to stimulate the heart to fire again, and begin beating. For example, it is envisioned that a system having a specifically-spaced neurostimulation assembly in or adjacent a select region of the epidural space can allow for an increased performance of CRT.

In yet another application of the methods and systems described herein, the presently described systems, methods, and associated apparatus and assemblies may be implanted into patients in order to both neurostimulate the patient to augment, provide therapeutic relief from, or therapeutically effect the CHF-related conditions described herein as well, while simultaneously acting as an ACID (an implantable cardioverter defibrillator, which is a device that is implanted in the chest to monitor for, and, if necessary, correct episodes of rapid heartbeat, such as in the instance of ‘ventricular tachycardia’ (VT) or ‘ventricular fibrillation (VF) in a patient). In this capacity, it would negate the concerns of having two or more different implants, as it is currently contra-indicated to implant an SCS (spinal cord stimulation) system or similar device in a patient having an AICD already implanted.

Finally, it is envisioned that the neurostimulation methods and systems disclosed herein may be useful in the treatment of vascular diseases, and other diseases associated with heart failure or poor performance of the heart itself. In example it is envisioned that peripheral vascular disease may be able to be treated by the selective placement of one or more neurostimulation devices on or near the thoracic spine area of a patient (e.g., T2, T3, or T4) via peripheral placement and stimulation.

The following examples are included to demonstrate preferred embodiments of the invention. It should be appreciated by those of skill in the art that the techniques disclosed in the examples which follow represent techniques discovered by the inventor(s) to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### EXAMPLES

### Prophetic Example 1

This trial is designed to provide preliminary evidence of the safety and efficacy of implanted neurostimulation as a therapy in chronic HF. Patients will preferably have relatively severe HF as depicted by NYHA functional class, LVEF (Left Ventricular Ejection Fraction), and their exercise capacity. All of the patients in the study will have a neurostimulation implant. The implant will be turned on in one group, will be turned off in others, and the study will include a randomized, cross-over design. Placebo control and exclusion control will be substantially similar to that described by Torre-Amione, G., et al., J. Am. Coll. Cardiol., Vol. 44 (6), pp. 1181-1186 (2004).

Methods

Patient selection. This is a single- or multi-center study with patients primarily drawn from HF (heart failure) clinics in and around the United States. Individuals ≧18 years of age with New York Heart Association (NYHA) (or equivalent) functional class III to IV, chronic HF, left ventricular ejection fraction (LVEF)_{—}40%, and 6-min walk distance<300 meters may be enrolled. Patients must have been receiving standard medical treatment. Doses of cardiac medications must have been stable for two weeks (in the case of beta-blockers, for three months) before randomization. The ethics committees at all participating institutions will approve the protocol. All patients participating will also give written informed consent.

Treatment regimen. Patients will receive active therapy (neurostimulation device implanted and turned on) or placebo (neurostimulation device implanted, but not turned on) on two consecutive days, followed by six monthly injections beginning two weeks later. Patients and clinicians will be blinded. There may also be an unblended operator who draws blood, treats the sample, and administers any appropriate treatment injections. This individual will not be involved in patient assessments, and unblinded staff will have no access to the treatment area.

Study procedures. After an initial screening/wash-out period of 2 to 14 days, there will be up to six months of treatment. Visit 10 (end-of-study) occurs 30 days after the last treatment. Patient assessments including NYHA functional class (or the equivalent), 6-min walk distance, Minnesota Living with Heart Failure Quality of Life (QoL) questionnaire, echo-Doppler, electrocardiogram, and blood and urine samples, among other appropriate tests as necessary, will be obtained at predetermined time points. Blinded measures of QTc (heart rate-corrected QT interval, the measure of the time between the start of the Q-wave and the end of the T-wave in the heart's electrical cycle) and QT dispersion (QTd) will be performed in patients with evaluable electrocardiograms. The QT intervals will measured from three consecutive beats as appropriate, such as in leads II and V4, then averaged, and corrected for heart rate using Fridericia's formula (or an equivalent); QTd will be determined by averaging the QT intervals from three consecutive beats in each lead and calculating the difference between the shortest and longest value.

Statistical analyses. The primary end points may include, but are not limited to, changes in 6-minute walk distance (6MWD) and NYHA functional classification. Secondary end points may include changes in cardiac function, all-cause mortality, all-cause hospitalizations, and Minnesota Living with Heart Failure Quality of Life (QoL) questionnaire score. Exploratory analyses will include a clinical composite score and change in QTc and QTd. For end points that had two or more post-treatment measurements, a repeated-measures analysis of variance (ANOVA) will be performed. For discrete end points, logistic models are used, as appropriate. Patients withdrawn from therapy for any reason (including death), will have the last observation carried forward. Cumulative survival curves for the risk of all-cause mortality and all-cause hospitalization will be constructed by the Kaplan-Meier (or similar) method; and differences between the curves will be tested for significance using the log-rank statistic, as warranted.

Primary and secondary efficacy parameters. Mean 6-minute walk distance (6MWD) for the implanted group receiving neurostimulation is to be measured (in meters) and recorded at baseline and at visit 10, noting any increase in an acceptable manner, such as percent increase. For the placebo group, the mean 6-minute walk distance at baseline will also be measured, and similarly the percentage increase at visit 10 will be determined, for purpose of comparison. By study end, the number of neurostimulation-treated patients (classified as to their CHF problem being treated) which have improved their NYHA (New York Heart Association) functional class will be determined, including the numbers which worsened, compared with the number of patients which exhibited some improvement, and the number that worsened in the placebo group.

Other and further embodiments utilizing one or more aspects of the inventions described above can be devised without departing from the spirit of Applicant's invention. For example, placement of the neurostimulation devices in locations along the spinal cord of the patient in locations not specifically detailed herein. Further, the various methods and embodiments of the treatment of cardiac dysfunctions such as heart failure (HF) using neurostimulation as described herein can be included in combination with each other to produce variations of the disclosed methods and embodiments. Discussion of singular elements can include plural elements and vice-versa.

The order of steps can occur in a variety of sequences unless otherwise specifically limited. The various steps described herein can be combined with other steps, interlineated with the stated steps, and/or split into multiple steps. Similarly, elements have been described functionally and can be embodied as separate components or can be combined into components having multiple functions.

The inventions have been described in the context of preferred and other embodiments and not every embodiment of the invention has been described. Obvious modifications and alterations to the described embodiments are available to those of ordinary skill in the art. The disclosed and undisclosed embodiments are not intended to limit or restrict the scope or applicability of the invention conceived of by the Applicants, but rather, in conformity with the patent laws, Applicants intend to fully protect all such modifications and improvements that come within the scope or range of equivalent of the following claims.

What is claimed is:

1. A method of treating a cardiac dysfunction in a human patient, the method comprising:
positioning at least one neurostimulation device adjacent a nerve of the patient; and
delivering an electrical stimulation pulse to the nerve of the patient for a period of time sufficient to result in an alleviation of the cardiac dysfunction.

2. A method of treating systolic heart failure in a patient, the method comprising:
positioning at least one neurostimulation device adjacent a peripheral nerve of a patient; and
delivering an electrical stimulation pulse to the peripheral nerve of the patient for a period of time sufficient to result in an alleviation of the systolic heart failure as indicated by decreased pulmonary resistance in the heart.

3. A method of treating diastolic heart failure in a patient, the method comprising:
positioning at least one neurostimulation device adjacent a peripheral nerve of a patient; and
delivering an electrical stimulation pulse to the peripheral nerve of the patient for a period of time sufficient to result in an alleviation of the diastolic heart failure as indicated by decreased blood pressure in the heart.

4. A method of treating arrhythmias in a patient, the method comprising:
positioning at least one neurostimulation device adjacent a peripheral nerve of a patient;
positioning at least one neurostimulation device adjacent a nerve in or near the brain of a patient; and
delivering an electrical stimulation pulse to the peripheral nerve and the nerve near the brain of the patient for a period of time sufficient to result in an alleviation of the arrhythmia as indicated by decreased arterial fibrillation and/or ventricular fibrillation.

5. A method of treating hypertension in a patient, the method comprising:
positioning at least one neurostimulation device adjacent a peripheral nerve of a patient;
positioning at least one neurostimulation device adjacent a nerve in or near the C2-C4 spinal region of a patient; and
delivering an electrical stimulation pulse to the peripheral nerve and the nerve near the C2-C4 spinal region of the patient for a period of time sufficient to result in an alleviation of the hypertension as indicated by improved blood pressure control in the patient.

6. A system for treating cardiac dysfunction in a patient, the system comprising:
a device including a stimulator adapted to deliver a stimulation signal for a heart failure event;
a plurality of sensors adapted to measure and provide information of a heart failure condition status at two separate times; and
a controller in communication with the stimulator and the plurality of sensors and adapted to use the measurements from the sensors to control the stimulator to modulate the electrical signal.

7. The system of claim 6, wherein the stimulator comprises at least one neural stimulator adapted to deliver a neural stimulation signal to a neural target for heart failure therapy.

8. The system of claim 6, wherein the information measured by the sensors includes an activity measurement, a blood pressure measurement, a pulmonary vascular resistance measurement, a heart rate variability (HRV) measurement, a heart rate turbulence (HRT) measurement, a respiration measurement, a heart sound measurement, a thoracic impedance measurement, and/or a metastolic blood flow measurement.

9. The system of claim 6, wherein the stimulator is a neurostimulator, and the device further comprises one or more devices consisting of an implantable cardioverter defibrillator (AICD), a bi-ventricular (BiV) device, and at least one feed-back loop, wherein the devices have electrical separateness.

10. The system of claim 9, wherein the feed-back loop is associated with bladder tone of the individual being treated, via a bladder stimulator.

11. The system of claim 9, wherein the fee-back loop is configured to perform a plurality of steps, including generating a variable tap coefficient signal.

12. A system for treating cardiac dysfunction in a patient, the system comprising:
means for applying an electrical stimulation to one or more nerves;
means for measuring one or more indicators of heart failure status in a patient;
means for monitoring the information obtained by the measurement means; and
means for adjusting the intensity and rate of application of electrical impulses using the information obtained by the measurement means.
